(19) United States
(12) Patent Application Publication
(10) Pub. No.: US 2026/0237072 A1
(43) Pub. Date: Aug. 13, 2026

(54) FUNCTIONAL RESPIRATORY IMAGING ANALYSIS OF SERALUTINIB FOR THE TREATMENT OF PULMONARY ARTERIAL HYPERTENSION (PAH)

(71) Applicant: GB002, INC., San Diego, CA (US)

(72) Inventors: Lawrence S. ZISMAN, San Diego, CA (US); Jean-Marie BRUEY, San Diego, CA (US); Robin OSTERHOUT, San Diego, CA (US); Benjamin LAVON, Kontich (BE); Patrick MUCHMORE, Kontich (BE)

(21) Appl. No.: 19/470,705

(22) PCT Filed: Mar. 28, 2024
(86) PCT No.: PCT/US2024/021986

§ 371 Date: Sep. 29, 2025

Related U.S. Application Data
(60) Provisional application No. 63/493,192, filed on Mar. 30, 2023.

(51) Int. Cl. G06T 7/00; A61B 5/00; A61B 5/091

(52) U.S. Cl. CPC G06T 7/0016; A61B 5/091; A61B 5/4848; G06T 2207/10081; G06T 2207/10088; G06T 2207/10104; G06T 2207/30061

(57) ABSTRACT

Methods for assessing the efficacy of Seralutinib treatment for PAH in subjects suffering from PAH are provided, as well as methods for increasing the ratio of the volume of distal pulmonary arteries relative to the volume of proximal pulmonary arteries (BV510ARatio) by administering to the subject an effective amount of Seralutinib. Also provided are methods for increasing the pulmonary vascular volume of small arterial vessels, as well as decreasing the dilation of larger pulmonary arterial vessels, in a subject in need thereof by administering to the subject an effective amount of Seralutinib.

### FIELD OF THE INVENTION

The present invention relates to functional respiratory imaging and analysis, in particular the functional respiratory imaging and analysis of patients suffering from Pulmonary Arterial Hypertension (PAH). The invention further relates to the treatment and treatment monitoring of said patients, in particular those being treated with a PDGFR inhibitor, a CSF1R inhibitor, a c-KIT kinase inhibitor or a combination thereof. In some embodiments, the PAH patients are being treated with Seralutinib.

### BRIEF SUMMARY

Respiratory conditions such as asthma, chronic obstructive pulmonary disease (COPD), cystic fibrosis (CF) and the like, are conditions that result in reduced gaseous exchange and may be evaluated using pulmonary function tests. Spirometry (the measuring of breath) is the most common pulmonary function test, measuring the amount (volume) and/or speed (flow) of air that can be inhaled and exhaled. However, in restrictive diseases (such as pulmonary fibrosis), breathing capacity is reduced and the measured values consequently distorted as a result of decreased lung compliance. Due to the limited availability and sensitivity of pulmonary function tests, evaluation of treatments and their effectiveness has proven challenging. Therefore, it is an object of the invention to provide improved methods for assessing the efficacy of a treatment of a respiratory condition.

Therefore, in one embodiment, the invention provides a method for assessing the efficacy of a treatment for a respiratory condition, the method comprising the steps of:
a) obtaining image data concerning two or more three-dimensional images of a patient's respiratory system, which images have been previously acquired during an assessment period;
b) calculating a specific three-dimensional structural model of the patient's respiratory system from each of the data obtained in step a); and
c) comparing the three-dimensional structural models of the patient's respiratory system for each of the image data obtained in step a) to assess the efficacy of a treatment for a respiratory condition.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1: Cartoon representation of the pulmonary arteries of healthy lung tissue and lung tissue of a patient suffering from PAH, showing the cross-sectional area of blood vessels throughout the lung.

FIG. 2: Graphical representation of a linear regression model of: (a) pulmonary artery compliance (PAC) vs BV510ARATIO; and (b) stroke volume (SV) vs BV510ARATIO (·=Seralutinib; =placebo).

FIG. 3: Plot of individual patient BV510ARatio (ratio of pulmonary arteries <5 mm2 in cross sectional area (BV5A) compared to pulmonary arteries >10 mm2 in cross sectional area (BV10A)) values at baseline and at week 24, after administration of Seralutinib or placebo.

FIG. 4: CASE STUDY 1: Lung images at (a) baseline and (b) week 24, of patient receiving placebo.

FIG. 5: CASE STUDY 2: Lung images at (a) baseline and (b) week 24, of patient receiving Seralutinib.

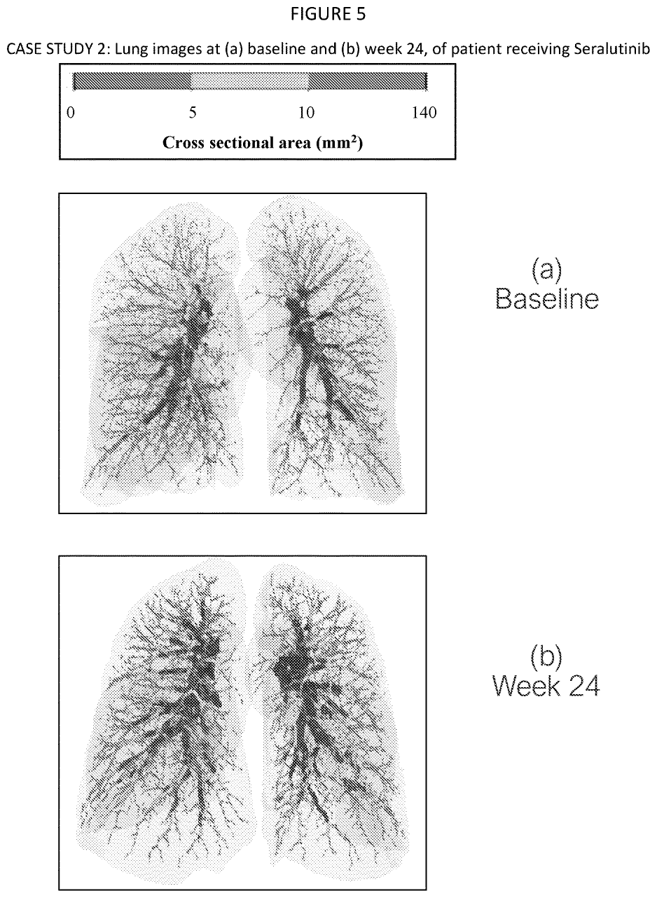

### DETAILED DESCRIPTION

Receptor tyrosine kinases are transmembrane polypeptides that regulate the regeneration, remodeling, development, and differentiation of cells. Among the receptor tyrosine kinases is the platelet derived growth factor receptor (PDGFR), which is associated with pulmonary diseases, tissue fibrosis, and solid tumors. Among the pulmonary diseases, pulmonary hypertension (PH) is a rare disorder of the pulmonary vasculature that is associated with high morbidity and mortality. The pathology of the disease includes plexiform lesions of disorganized angiogenesis and abnormal neointimal cellular proliferation, which obstruct blood flow through the pulmonary arterioles.

Seralutinib, (chemical name N-{3-[(1S)-1-{[6-(3,4-dimethoxyphenyl) pyrazin-2-yl]amino}ethyl]phenyl}-5-methylpyridine-3-carboxamide and also known as GB002), is a highly potent and selective inhibitor of PDGFRα and PDGFRβ signaling, under clinical development as an inhaled treatment for pulmonary arterial hypertension (PAH). Seralutinib has the following structure:

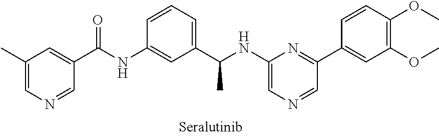

An amorphous form of Seralutinib was described in U.S. Pat. Nos. 9,815,815 and 10,231,966, and in a spray-dried powder formulation in U.S. Pat. No. 9,925,184.

Therapies available for PAH treatment include therapeutically active compounds, as noted herein and/or known in the art, include, but are not limited to, prostanoids, endothelin antagonists, cytoplasmic kinase inhibitors, receptor kinase inhibitors, endothelin receptor antagonists, e.g., ambrisentan, bosentan, and sitaxentan, PDE5 (PDE-V) inhibitors, e.g., sildenafil, tadalafil, and vardenafil, calcium channel blockers, e.g., amlodipine, felodipine, verapamil, diltiazem, and menthol, prostacyclin, treprostinil, iloprost, beraprost, nitric oxide, oxygen, heparin, warfarin, diuretics, digoxin, cyclosporins, e.g., cyclosporin A, CTLA4-Ig, antibodies such as ICAM-3, anti-IL-2 receptor (Anti-Tac), anti-CD45RB, anti-CD2, anti-CD3 (OKT-3), anti-CD4, anti-CD80, anti-CD86, agents blocking the interaction between CD40 and gp39, such as antibodies specific for CD40 and/or gp39, i.e., CD 154, fusion proteins constructed from CD40 and gp39 (CD40 1 g and CD8gp39), inhibitors, such as nuclear translocation inhibitors, of NF-kappa B function, such as deoxyspergualin (DSG), cholesterol biosynthesis inhibitors such as HMG CoA reductase inhibitors (lovastatin and simvastatin), non-steroidal anti-inflammatory drugs (NSAIDs) such as ibuprofen, aspirin, acetaminophen, leflunomide, deoxyspergualin, cyclooxygenase inhibitors such as celecoxib, steroids such as prednisolone or dexamethasone, gold compounds, beta-agonists such as salbutamol, LABAs such as salmeterol, leukotriene antagonists such as montelukast, antiproliferative agents such as methotrexate, FK506 (tacrolimus, Prograf), mycophenolate mofetil, cytotoxic drugs such as azathioprine, VP-16, etoposide, fludarabine, doxorubin, adriamycin, amsacrine, camptothecin, cytarabine, gemcitabine, fluorodeoxyuridine, melphalan and cyclophosphamide, antimetabolites such as methotrexate, topoisomerase inhibitors such as camptothecin, DNA alkylators such as cisplatin, kinase inhibitors such as sorafenib, microtubule poisons such as paclitaxel, TNF-α inhibitors such as tenidap, anti-TNF antibodies or soluble TNF receptor, hydroxy urea and rapamycin (sirolimus or Rapamune) or derivatives thereof.

Similarly, the terms “effective amount” or “pharmaceutically effective amount” is a quantity sufficient to achieve a desired therapeutic and/or prophylactic effect, e.g., an amount which results in the prevention of, or a decrease in, the symptoms associated with a disease that is being treated. The amount of Seralutinib administered to the subject will depend on the type and severity of the disease and on the characteristics of the individual, such as general health, age, sex, body weight and tolerance to drugs. The skilled artisan will be able to determine appropriate dosages depending on these and other factors.

Representative diseases or conditions that may be treated with Seralutinib include, but are not limited to, PAH, primary PAH, idiopathic PAH, heritable PAH, refractory PAH, BMPR2, ALK1, endoglin associated with hereditary hemorrhagic telangiectasia, endoglin not associated with hereditary hemorrhagic telangiectasia, drug-induced PAH, and toxin-induced PAH, PAH associated with or secondary to one or more of systemic sclerosis, mixed connective tissue disease, cancer, refractory cancer, metastatic cancer, neoplasia, hypoplasia, hyperplasia, dysplasia, metaplasia, prosoplasia, desmoplasia, angiogenic disease, pulmonary function disorders, cardiovascular function disorders, HIV infection, hepatitis, portal hypertension, pulmonary hypertension, congenital heart disease, hypoxia, chronic hemolytic anemia, newborn persistent pulmonary hypertension, pulmonary veno-occlusive disease (PVOD), pulmonary capillary hemangiomatosis (PCH), left heart disease pulmonary hypertension, systolic dysfunction, diastolic dysfunction, valvular disease, lung disease, interstitial lung disease, pulmonary fibrosis, schistosomiasis, chronic obstructive pulmonary disease (COPD), sleep-disordered breathing, alveolar hypoventilation disorders, chronic exposure to high altitude, developmental abnormalities, chronic thromboembolic pulmonary hypertension (CTEPH), pulmonary hypertension with unclear multifactorial mechanisms, hematologic disorders, myeloproliferative disorders, splenectomy, systemic disorders, sarcoidosis, pulmonary Langerhans cell histiocytosis, lymphangioleiomyomatosis, neurofibromatosis, vasculitis, metabolic disorders, glycogen storage disease, Gaucher disease, thyroid disorders, tumoral obstruction, fibrosing mediastinitis, and chronic renal failure on dialysis; and diseases such as pulmonary hypertension, congenital heart disease, hypoxia, chronic hemolytic anemia, newborn persistent pulmonary hypertension, pulmonary veno-occlusive disease (PVOD), pulmonary capillary hemangiomatosis (PCH), left heart disease pulmonary hypertension, systolic dysfunction, diastolic dysfunction, valvular disease, lung disease, interstitial lung disease, pulmonary fibrosis, schistosomiasis, chronic obstructive pulmonary disease (COPD), sleep-disordered breathing, alveolar hypoventilation disorders, chronic exposure to high altitude, developmental abnormalities, chronic thromboembolic pulmonary hypertension (CTEPH), pulmonary hypertension with unclear multifactorial mechanisms, hematologic disorders, myeloproliferative disorders, splenectomy, systemic disorders, sarcoidosis, pulmonary Langerhans cell histiocytosis, lymphangioleiomyomatosis, neurofibromatosis, vasculitis, metabolic disorders, glycogen storage disease, Gaucher disease, thyroid disorders, tumoral obstruction, fibrosing mediastinitis, immunological and inflammatory diseases, hyperproliferative diseases, renal and kidney diseases, bone remodeling diseases, metabolic diseases, vascular diseases, and chronic renal failure on dialysis.

In one aspect, the disease or condition is pulmonary arterial hypertension (PAH), and a therapeutically effective amount of the crystalline form of Seralutinib is administered to subject in need thereof. In specific embodiments, the disease or condition is PAH, primary PAH, idiopathic PAH, heritable PAH, refractory PAH, drug-induced PAH, toxin-induced PAH, or PAH with secondary diseases.

### Functional Respiratory Imaging

Traditional lung function measurements, such as FEV₁ or FVC, provide information about the general condition of the entire lung but do not provide details of specific regions of the lung. Regional information is important to understand the pathophysiology of the individual patient and provide guidance for optimal disease treatment. Functional respiratory imaging (FRI) is a non-invasive measurement of the patient-specific respiratory system.

FRI initially acquires low dose, high-resolution computed tomography (HRCT) scans of the patient. Measurements are typically performed on the segmented 3-dimensional geometries derived from these scans and then computational fluid dynamics (CFD) quantifies airflow and exposure to inhaled particles.

This results in a set of biomarkers (such as lung, blood vessel, nodule and airway volumes, airway resistance and Internal airflow distribution, ventilation mapping and perfusion reserve), which collectively evaluate exposure, structure and function of the lungs and airway. This provides critical information on lung disease stage and response (or non-response) to treatment, thereby guiding clinical decision making and overall improving patient care. Use of FRI biomarkers is scalable and easy to implement and is an important addition to the toolkit of research and clinical practice in respiratory diseases.

Determining a respiratory-type condition, the treatment of a respiratory-type condition, and the monitoring of the treatment of a respiratory-type condition, for a patient suffering from a respiratory-type condition, (such as hypercapnic chronic obstructive pulmonary disease (COPD), asthma, cystic fibrosis (CF), amyotrophic lateral sclerosis (ALS), myotonic dystrophy (Steinert's disease), Duchenne muscular dystrophy, Acid maltase deficiency and Emery-Dreifuss myopathy) using data concerning three-dimensional images of the respiratory system of the patient, has been previously described. See for example U.S. Pat. No. 11,109,830, Jan De Backer, “Method for determining a respiratory condition based on functional respiratory imaging”; U.S. Pat. No. 8,886,500, Jan De Backer, “Method for determining treatments using patient-specific lung models and computer methods”; or US-2012-0072193, Jan De Backer, “Method for determining treatments using patient-specific lung models and computer methods”.

For example, in asthma and COPD patients, FRI was used for phenotyping to determine the responder/non-responder phenotype and to evaluate various therapeutic interventions.

Described herein are methods for assessing the efficacy of treatment of a patient suffering from PAH. In some embodiments, the methods are for assessing the efficacy of Seralutinib treatment of a patient suffering from PAH.

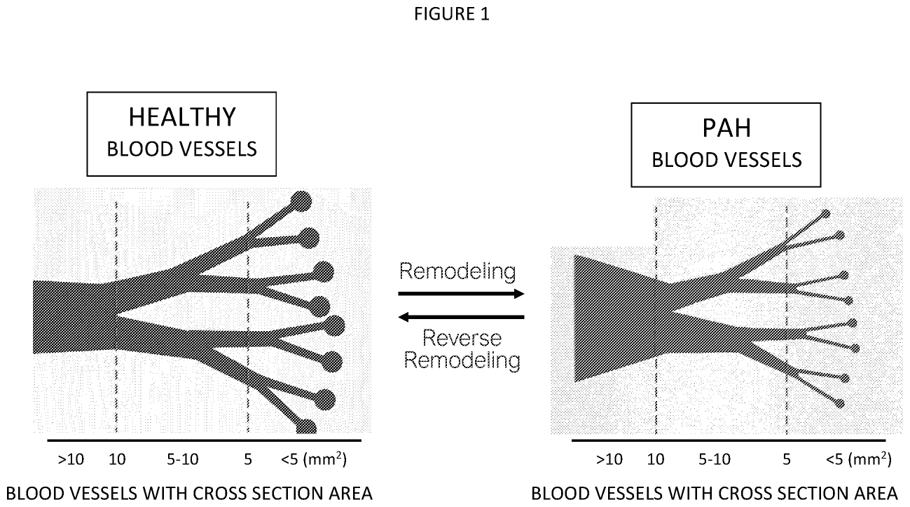

FIG. 1 presents a graphical representation of the pulmonary arteries of healthy lung tissue and lung tissue of a patient suffering from PAH, showing the cross-sectional area of blood vessels throughout the lung. Blood vessel volume of pulmonary arteries with a cross-sectional area (CSA):

$$B V 5 A = C S A \text{<5} {mm}^{2} ;$$

$$B V 5 - 10 A = C S A 5 - 10 {mm}^{2} ; and$$

$$B V 10 A = C S A \text{>10} {mm}^{2} .$$

In healthy lung tissue the ratio of BV5A to BV10A (BV510ARatio) is higher than in diseased tissue.

The images may have been previously acquired using any method of the art. Such methods include magnetic resonance imaging, positron emission tomography and computer tomography (CT) imaging and the like.

The “respiratory system” refers to the intra- and extra thoracic airways and the lungs. In some instances, the images are acquired at total lung capacity (TLC), the lung level attained after a deep inhalation.

The present invention is further illustrated by the following examples, which should not be construed as limiting in any way.

### EXAMPLES

The various embodiments described above can be combined to provide further embodiments. All the U.S. patents, U.S. patent application publications, U.S. patent applications, foreign patents, foreign patent applications and non-patent publications referred to in this specification and/or listed in the Application Data Sheet are incorporated herein by reference, in their entirety. Aspects of the embodiments can be modified, if necessary, to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

All publications, patents, and patent applications mentioned in this specification are herein incorporated by reference to the same extent as if each individual publication, patent, or patent application was specifically and individually indicated to be incorporated by reference.

### EXAMPLES

### Example 1

### Clinical Study to Investigate Pulmonary Vascular Remodeling in Pulmonary Arterial Hypertension (PAH) Patients Treated with Seralutinib

Typically, pulmonary vascular volume of small arterial vessels is decreased in PAH, leading to dilation of larger vessels, (referred to as remodeling), as demonstrated by the graphic shown in FIG. 1.

A clinical study was undertaken to examine the effect of Seralutinib on pulmonary arterial blood vessel volume distribution in PAH patients. Seralutinib was administered twice daily (BID) for 24 weeks, via dry powder inhalation, to PAH patients (WHO Group I, functional class II or III). Thin-section, volumetric non-contrast chest CTs were obtained (at baseline and week 24, minimum of 64 slices), followed by automated pulmonary vascular segmentation to evaluate the reverse remodeling potential of seralutinib.

From the scans, paired inspiratory-expiratory high-resolution non-contrast computer tomography studies were performed and the lobar tissue, airways and vasculature were reconstructed. Vasculature volumes are provided as a total blood volume (BV), and as BVX values, which divide the pulmonary blood volume depending on the size vessel in which the blood is contained, wherein:

$$B V 5 refers to vessels with a C S A \text{<5} {mm}^{2} ;$$

$$B V 5 - 10 refers to vessels with a C S A 5 - 10 {mm}^{2} ;$$

$$B V 10 refers to vessels with a C S A \text{>10} {mm}^{2} ; and$$

$$B V 10 Ratio = B V 5 / B V 10.$$

Blood vessel volumes (BVV) of pulmonary arteries with a CSA<5 mm² (BV5A) and >10 mm² (BV10A) were calculated, and the BV5A to BV10A ratio (BV510ARATIO) was used to express relative redistribution of pulmonary arterial blood vessel volume. Linear regression was used to model the treatment effect.

Baseline and Week 24 CTs were obtained in 19 subjects (7 receiving seralutinib, 12 receiving placebo), mean age 49±12 years, 18 were female, as presented in Tables 1 and 2. All subjects were receiving two or three approved PAH-specific medications.

TABLE 1
Patient Demographics

| | | |
|---|---|---|
| Patient | Baseline | Follow-up |

| n | | 21 | | 19 | |
|---|---|---|---|---|---|
| Treatment (%) | Seralutinib | 8 | (38.1) | 7 | (36.8) |
| | Placebo | 13 | (61.9) | 12 | (63.2) |
| Age (mean (SD)) | | 49.48 | (11.47) | 49.26 | (12.07) |
| Gender (%) | F | 20 | (95.2) | 18 | (94.7) |
| | M | 1 | (4.8) | 1 | (5.3) |

| | | | | |
|---|---|---|---|---|
| BMI (mean (SD)) | 30.84 | (7.36) | 30.42 | (7.59) |

TABLE 2
Patient Characteristics

| | Baseline | | | Change | | |
|---|---|---|---|---|---|---|
| Treatment | PVR | MPAP | PAC | PVR | MPAP | PAC |
| Seralutinib | 533 | 44 | 1.63 | −52 | −8 | 0.85 |
| Seralutinib | 970 | 48 | 1.00 | −235 | 3 | 0.31 |
| Seralutinib | 836 | 49 | 0.90 | −139 | −2 | 0.11 |
| Seralutinib | 591 | 52 | 1.72 | −171 | −3 | 0.32 |
| Seralutinib | 645 | 59 | 1.08 | NA | NA | NA |
| Seralutinib | 408 | 31 | 2.87 | −159 | −4 | 0.65 |
| Seralutinib | 516 | 34 | 1.84 | −11 | −3 | 0.24 |
| Placebo | 650 | 41 | 1.38 | −193 | −2 | 0.25 |
| Placebo | 560 | 43 | 1.89 | 727 | 32 | −1.16 |
| Placebo | 433 | 45 | 1.82 | 283 | 20 | −0.82 |
| Placebo | 660 | 44 | 1.48 | −115 | 5 | 0.25 |
| Placebo | 533 | 48 | 1.46 | −170 | 1 | 0.10 |
| Placebo | 412 | 44 | 1.73 | −1 | 1 | 0.21 |
| Placebo | 665 | 48 | 1.55 | −200 | −12 | 0.72 |
| Placebo | 625 | 64 | 1.07 | 172 | −5 | 0.25 |
| Placebo | 729 | 45 | 1.16 | −198 | −6 | 0.39 |
| Placebo | 551 | 48 | 1.61 | 91 | 8 | −0.24 |
| Placebo | 614 | 55 | 1.23 | 169 | 0 | −0.12 |
| Placebo | 994 | 51 | 1.43 | −69 | −7 | −0.13 |

PAC = pulmonary arterial compliance

Correlation Analysis provided Change (from baseline to week 24) in blood volume arterial segment vs. Change in Clinical Parameters, as shown in Table 3 below:

TABLE 3

| | | BNPPRONT | CARDOUT | MPAP | MRAP | PAC | PVR |
|---|---|---|---|---|---|---|---|
| BV5 | 1.3 | −0.29 | −0.08 | −0.06 | 0.1 | −0.03 | −0.03 |
| Arterial | 2.48 | −0.25 | 0.14 | −0.52 | −0.35 | 0.51 | −0.17 |
| Segments | 4.15 | −0.39 | 0.4 | −0.63 | −0.32 | 0.72 | −0.54 |
| BV10 | 24.75 | 0.38 | −0.34 | 0.32 | 0.23 | −0.61 | 0.2 |
| Arterial | 35.63 | 0.23 | −0.23 | 0.69 | 0.45 | −0.63 | 0.45 |
| Segments | 48.52 | −0.12 | −0.05 | 0.26 | 0.5 | −0.27 | −0.21 |

The regression model effect estimates were as shown in Table 4 below:

TABLE 4

| | | conf. | conf. | std. | | p. |
|---|---|---|---|---|---|---|
| Parameter | estimate | low | high | error | statistic | value |
| BV510ARATIO | 0.845 | 0.105 | 1.585 | 0.349 | 2.420 | 0.028 |
| BV510RATIO | 0.843 | −0.034 | 1.721 | 0.414 | 2.037 | 0.059 |
| BV10APRA | −2.711 | −5.693 | 0.271 | 1.406 | −1.928 | 0.072 |
| BV10PR | −2.180 | −4.896 | 0.536 | 1.281 | −1.702 | 0.108 |
| BV5APRA | 2.625 | −1.231 | 6.480 | 1.819 | 1.443 | 0.168 |
| BV5PR | 1.835 | −1.646 | 5.315 | 1.642 | 1.117 | 0.280 |

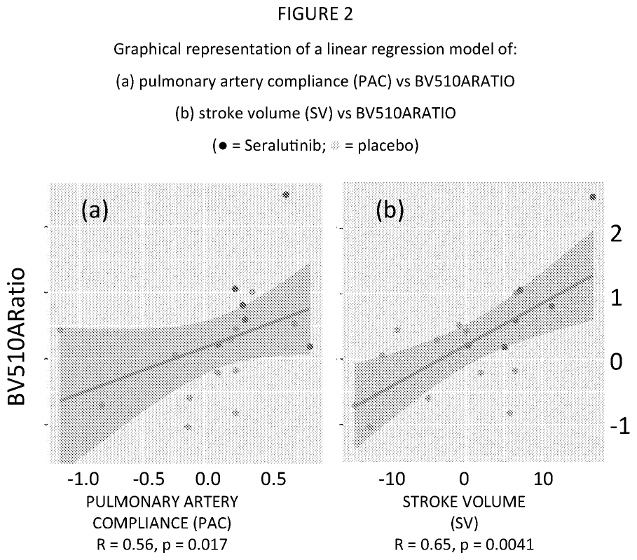

BV510A Ratio was significantly higher in the seralutinib group compared to placebo (p=0.028) and correlated with stroke volume (R=0.65, p=0.0041) and pulmonary artery compliance (R=0.56, p=0.017). FIG. 2 confirms BV510ARatio correlates with hemodynamics.

FIG. 2(a) shows a linear regression model, adjusted for baseline values and treatment arm, of pulmonary artery compliance (PAC) vs BV510ARATIO (the Ratio of pulmonary arteries <5 mm² in cross sectional area (BV5A) compared to pulmonary arteries >10 mm² in cross sectional area (BV10A)). (·=Seralutinib; ·=placebo).

FIG. 2(b) shows a linear regression model adjusted for baseline values and treatment arm, of stroke volume (SV) vs BV510ARATIO (the Ratio of pulmonary arteries <5 mm² in cross sectional area (BV5A) compared to pulmonary arteries >10 mm2 in cross sectional area (BV10A)). (·=Seralutinib; ∘=placebo).

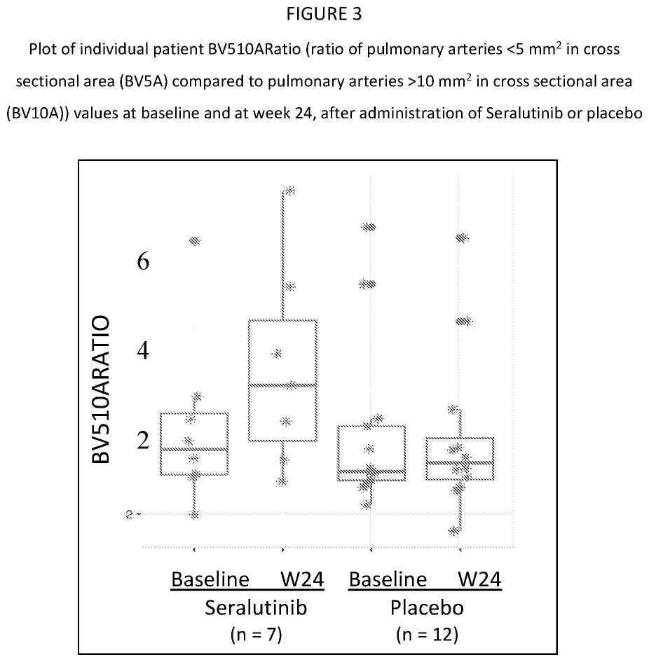

The least squares mean difference value of the BV510ARatio (the ratio of pulmonary arteries <5 mm² in cross sectional area (BV5A) compared to pulmonary arteries >10 mm² in cross sectional area (BV10A)) increased 0.845 (95% Cl=0.105, 1.585, with a p-value of 0.028), i.e. Seralutinib increases BV510ARatio (see FIG. 3, which shows a plot of individual patient BV510ARatio values at baseline and at week 24, after administration of Seralutinib or placebo).

BV510A Ratio correlates with important measures of RV-PA coupling as measured by pulmonary artery compliance and cardiopulmonary hemodynamics (i.e., Stroke Volume).

For subjects with PAH on dual or triple PAH therapies, adding seralutinib led to a significant redistribution of pulmonary arterial blood vessel volume to more distal vessels, suggesting a reverse remodeling effect of seralutinib in PAH.

### Example 2

### Case Studies

TWO case studies were undertaken, as described below.

### Case Study 1 (Placebo)

Case study 1 was of 24-year-old female with WHO functional class II, idiopathic PAH receiving approved PAH-specific medications (phosphodiesterase type 5i and Prostacyclins/PRA).

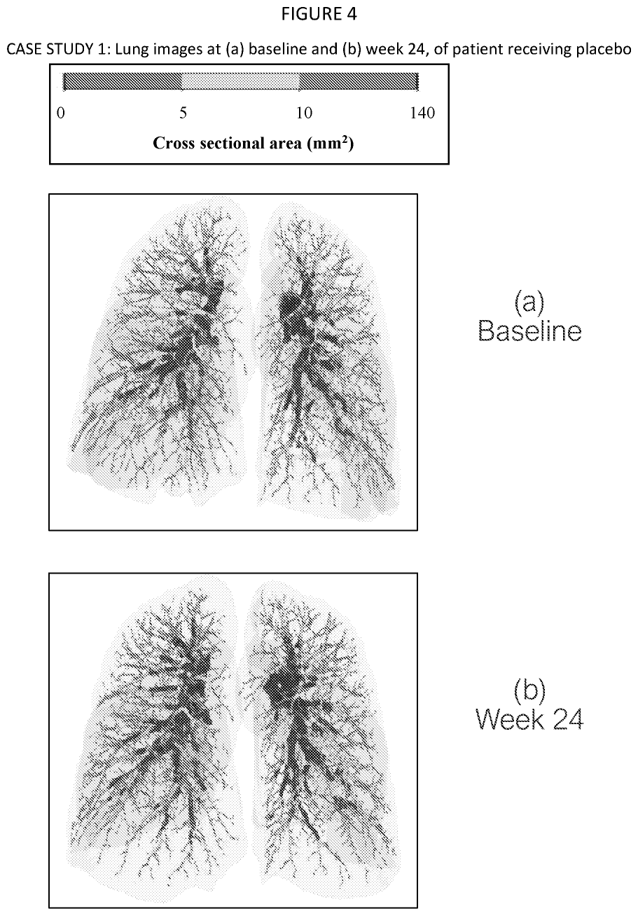

FIG. 4 shows lung images at baseline and week 24 after being administered placebo twice daily (BID) for 24 weeks, via dry powder inhalation, demonstrating:
6% Decrease in BV5APRA (red areas);
5% Increase in BV10APRA (blue areas);
Decrease in BV5A/BV10A ratio; and
283 dyne increase in PVR coincident with arterial volume shifts.

$$B V A 5 P R A = proportion of pulmonary arteries \text{<5} {mm}^{2} in cross sectional area compared to all arteries .$$

$$B V A 10 P R A = proportion of pulmonary arteries \text{>10} {mm}^{2} in cross sectional area compared to all arteries .$$

| PVR | | | | ΔBV5A | ΔBV10A | |
|---|---|---|---|---|---|---|
| base- | PVR | 6MWD | NTproBNP | PRA | PRA | ΔBV510A |
| line | change | change | change | (%)* | (%)* | RATIO |
| 433 | 283 | −34 | 118 | −6.6 | 5.0 | −0.70 |

### Case Study 2 (Seralutinib)

Case study 2 was of a 58-year-old female with WHO functional class II idiopathic PAH+severe rheumatoid arthritis receiving approved PAH Triple Therapy (ERA+PDE-5i+PRA).

FIG. 4 shows lung images at baseline and week 24 after being administered seralutinib twice daily (BID) for 24 weeks, via dry powder inhalation, demonstrating:
6% Increase in BV5APRA (red areas);
8% Decrease in BV10APRA (blue areas);
Increase in BV5A/BV10A ratio; and 10
Improvement in PVR coincident with arterial volume shifts.

| | | | | BV5A | BV10A | |
|---|---|---|---|---|---|---|
| PVR | PVR | 6MWD | NTproBNP | PRA | PRA | BV510A |
| baseline | change | change | change | (%)* | (%)* | RATIO |
| 408 | −159 | −48 | 41 | +5.6 | −7.7 | +2.5 |

This application claims the benefit of priority to U.S. Application No. 63/493,192, filed Mar. 30, 2023, which application is hereby incorporated by reference in its entirety.

1. A method for assessing the efficacy of Seralutinib treatment for PAH in a subject suffering from PAH, comprising the steps of:
a) obtaining data concerning a pre-treatment three-dimensional image of a respiratory system of the patient, and a post-treatment three-dimensional image of the respiratory system of the subject;
b) calculating a specific three-dimensional structural model of a lung structure of the patient from each of the pre- and post-treatment image data obtained in step a);
c calculating a specific three-dimensional structural model of an airway structure of the patient from each of the pre- and post-treatment image data obtained in step a);
d) calculating a patient-specific three-dimensional structural model of a lobar structure of the subject from each of the pre- and post-treatment lung structure models obtained in step b);
e) modeling by a computer, air flow through the airway structure at pre- and post-treatment states, using the respective pre- and post-treatment models of the airway structure and lobar structure of the subject obtained in steps c) and d);
f) modeling by a computer, structural behavior of the airway structure and the interaction with the air flow at pre- and post-treatment states, using the respective pre- and post-treatment models of the airway structure and lobar structure of the subject obtained in steps c) and d); and
g) comparing the modeled air flow pre- and post-treatment and comparing the structural behavior pre- and post-treatment to determine the efficacy of the Seralutinib treatment, wherein an efficacious treatment is one that decreases airway resistance, thereby determining the efficacy of the seralutinib treatment.

2. A method of reverse remodeling of pulmonary vasculature in a subject in need thereof, comprising administering to the subject an effective amount of Seralutinib.

3. A method of increasing the ratio of the volume of distal pulmonary arteries relative to the volume of proximal pulmonary arteries (BV510ARatio) in a subject in need of an increased BV510ARatio, comprising administering to the subject an effective amount of Seralutinib.

4. The method of claim 3 wherein the increase in the BV510ARatio is at least 0.5.

5. The method of claim 3 wherein the increase in the BV510ARatio is between 0.5 and 1.0.

6. The method of claim 3 wherein the increase in the BV510ARatio is at least 0.8.

7. A method of increasing the pulmonary vascular volume of small arterial vessels in a subject in need thereof, comprising administering to the subject an effective amount of Seralutinib.

8. A method of decreasing the dilation of larger pulmonary arterial vessels in a subject in need thereof, comprising administering to the subject an effective amount of Seralutinib.
